(19) United States
(12) Patent Application Publication
(10) Pub. No.: US 2026/0130578 A1
(43) Pub. Date: May 14, 2026

(54) BRONCHOSCOPE PATH ASSISTANCE SYSTEM, POSITIONING MOUTHPIECE AND BRONCHOSCOPE PATH DISPLAY DEVICE

(71) Applicant: HUKUI BIOTECHNOLOGY CO., LTD., Taipei City (TW)

(72) Inventors: SHIH-CHE LO, Taipei City (TW); MING-KUN CHAN, Taipei City (TW); YI-MING CHU, Taipei City (TW)

(73) Assignee: HUKUI BIOTECHNOLOGY CO., LTD., Taipei City (TW)

(21) Appl. No.: 19/032,131

(22) Filed: Jan. 20, 2025

Related U.S. Application Data
(60) Provisional application No. 63/653,242, filed on May 30, 2024.

(30) Foreign Application Priority Data
Oct. 31, 2024 (TW) 113141787

(51) Int. Cl. A61B 1/00; A61B 1/267

(52) U.S. Cl. CPC A61B 1/00154; A61B 1/00039; A61B 1/00045; A61B 1/2676

(57) ABSTRACT

A bronchoscope path assistance system includes: a bronchoscope, having a camera device on one end thereof, for generating an image signal; a mouthpiece, having an opening at a center thereof; a positioning device, disposed on the mouthpiece, for detecting a relative movement between the bronchoscope and the mouthpiece to issue a positioning signal; a memory device, for storing path information; a processing device, for generating a composite image signal according to the image signal, the positioning signal and the path information; a display unit; and an input unit. The bronchoscope path assistance system of the present disclosure is adapted for treatment, inspection or sampling of bronchi. The present disclosure further relates to a positioning mouthpiece and a bronchoscope path display device.

### CROSS-REFERENCE TO RELATED APPLICATION

This non-provisional application claims priority under 35 U.S.C. § 119(a) on Patent Application No(s). 113141787 filed in Taiwan, R.O.C. on Oct. 31, 2024, the entire contents of which are hereby incorporated by reference.

This non-provisional application claims priority under 35 U.S.C. § 119(e) on U.S. provisional Patent Application No(s). 63/653,242 filed on May 30, 2024, the entire contents of which are hereby incorporated by reference.

### BACKGROUND OF THE INVENTION

### 1. Field of the Invention

The present disclosure relates to a bronchoscope path assistance system, and in particular to a bronchoscope path assistance system capable of generating a composite image signal including an assistance positioning information. The present disclosure further relates to a positioning mouthpiece applicable in the bronchoscope path assistance system above. The present disclosure yet further relates a bronchoscope path display device operable to receive a positioning signal so as to display a position of a bronchoscope.

### 2. Description of the Related Art

Paths in bronchi of human lungs are extremely complex. Conventionally, treatment, inspection or sampling performed by a physician on lungs of a patient or an inspection subject by using a bronchoscope is dependent to a vast extent on knowledge and clinical experience of the physical regarding paths of bronchi.

However, even for a physician with abundant knowledge and clinical experience, rare occasions of loss of direction and inability to determine a lung area where the bronchoscope is currently located may still take place. At this point, the physician needs to back the bronchoscope to a previous or even earlier branch of a bronchus in order to correctly re-enter a desired lung area. The situation above causes additional risks for a patient or an inspection subject, and also reduces efficiency of treatment, inspection or sampling of bronchi.

### BRIEF SUMMARY OF THE INVENTION

There is a need for improvement for a conventional bronchoscope. Therefore, it is one object of the present disclosure to provide a bronchoscope path assistance system, so as to enhance efficiency of lung treatment, inspection of sampling implemented by a bronchoscope.

To achieve the above and other objects, the present disclosure provides a bronchoscope path assistance system including: a bronchoscope, having a camera device on one end thereof, for generating an image signal; a mouthpiece, having an opening at a center thereof, adapted to guide the bronchoscope to pass through a mouth of an inspection subject via the opening; a positioning device, disposed on the mouthpiece, for detecting a relative movement between the bronchoscope and the mouthpiece to issue a positioning signal; a memory device, for storing path information associated with branch paths of bronchi; a processing device, for receiving the image signal and the positioning signal and accessing the path information to generate a composite image signal according to the image signal, the positioning signal and the path information, wherein the composite image signal includes the image signal and an assistance positioning information generated according to the positioning signal and the path information; a display unit, for receiving and displaying the composite image signal; and an input unit, for receiving a determined path information input by a user, wherein, after the user inputs the determined path information, the processing device generates a new assistance positioning information according to the determined path information, the positioning signal and the path information.

In one embodiment of the present disclosure, the positioning device of the bronchoscope path assistance system includes a plurality of positioning cameras, the bronchoscope has a plurality of equidistant positioning marks, and the positioning device detects the relative movement between the bronchoscope and the mouthpiece according to images of the positioning marks captured by the plurality of positioning cameras.

In one embodiment of the present disclosure, the positioning device of the bronchoscope path assistance system includes four positioning cameras, which are located on upper, lower, left and right sides of the opening of the mouthpiece, respectively.

In one embodiment of the present disclosure, the positioning device of the bronchoscope path assistance system includes a plurality of positioning rollers; during use, the plurality of positioning rollers are in contact with the bronchoscope, and the positioning device detects the relative movement between the bronchoscope and the mouthpiece according to rotation magnitudes of the plurality of positioning rollers.

In one embodiment of the present disclosure, the positioning device of the bronchoscope path assistance system includes four positioning rollers, which are located on upper, lower, left and right sides of the opening of the mouthpiece, respectively.

In one embodiment of the present disclosure, the positioning device of the bronchoscope path assistance system further includes: a positioning rotor, disposed on a guide rail on the mouthpiece, wherein the positioning rotor and the guide rail are disposed around the opening of the mouthpiece, and the positioning rotor has thereon a plurality of holes in an annular arrangement and is provided with a plurality of elastic clamping devices in an interference fit with the bronchoscope; and an encoder, disposed at a position relative to the plurality of holes on the positioning rotor, and detecting a rotation direction and a rotation angle between the bronchoscope and the mouthpiece according to a direction and an magnitude of a displacement of the plurality of holes.

In one embodiment of the present disclosure, the display unit and the input unit are integrated into a touch screen.

In one embodiment of the present disclosure, the memory device, the processing device, a display unit and the input unit are integrated into a tablet computer.

In one embodiment of the present disclosure, a tip of the bronchoscope of the bronchoscope path assistance system has a tip positioning device. The tip positioning device is for generating a tip positioning signal, and transmitting the tip positioning signal to the processing device, wherein the processing device generates the composite image signal further according to the tip positioning signal.

In one embodiment of the present disclosure, the tip positioning device is selected from the group consisting of accelerometer and gyroscope.

In one embodiment of the present disclosure, the mouthpiece of the bronchoscope path assistance system has a plurality of initial orientation positioning marks located near the opening of the mouthpiece. When the camera device on one end of the bronchoscope is about to pass through the mouthpiece, the image signal generated by the camera device is to include the initial orientation positioning marks, and the processing device further analyzes the image signal including the plurality of initial orientation positioning marks to determine an initial orientation and generates the composite image signal further according to the initial orientation.

To achieve the above and other objects, the present disclosure further provides a positioning mouthpiece including: a mouthpiece, having an opening at a center thereof, adapted to guide a bronchoscope to pass through a mouth of an inspection subject via the opening; and a positioning device, disposed on the mouthpiece, for detecting a relative movement between the bronchoscope and the mouthpiece to issue a positioning signal.

In one embodiment of the present disclosure, the positioning device of the positioning mouthpiece includes four positioning cameras, which are located on upper, lower, left and right sides of the opening of the mouthpiece, respectively.

In one embodiment of the present disclosure, the positioning device of the positioning mouthpiece includes a plurality of positioning rollers. During use, the plurality of positioning rollers are in contact with the bronchoscope, and the positioning device detects the relative movement between the bronchoscope and the mouthpiece according to rotation magnitudes of the plurality of positioning rollers.

In one embodiment of the present disclosure, the positioning device of the positioning mouthpiece includes four positioning rollers, which are located on upper, lower, left and right sides of the opening of the mouthpiece, respectively.

In one embodiment of the present disclosure, the positioning device of the positioning mouthpiece further includes: a positioning rotor, disposed on a guide rail on the mouthpiece, wherein the positioning rotor and the guide rail are disposed around the opening of the mouthpiece, and the positioning rotor has thereon a plurality of holes in an annular arrangement and is provided with a plurality of elastic clamping devices in an interference fit with the bronchoscope; and an encoder, disposed at a position relative to the plurality of holes on the positioning rotor, and detecting a rotation direction and a rotation angle between the bronchoscope and the mouthpiece according to a direction and an magnitude of a displacement of the plurality of holes.

In one embodiment of the present disclosure, the mouthpiece of the positioning mouthpiece has a plurality of initial orientation positioning marks located near the opening of the mouthpiece.

To achieve the above and other objects, the present disclosure further provides a bronchoscope path display device including: a bronchoscope, having a camera device on one end thereof, for generating an image signal; a memory device, for storing path information associated with branch paths of bronchi; a processing device, for receiving the image signal and a positioning signal and accessing the path information to generate a composite image signal according to the image signal, the positioning signal and the path information, wherein the composite image signal includes the image signal and an assistance positioning information generated according to the positioning signal and the path information; a display unit, for receiving and displaying the composite image signal; and an input unit, for receiving a determined path information input by a user, wherein, after the user inputs the determined path information, the processing device generates a new assistance positioning information according to the determined path information, the positioning signal and the path information.

In one embodiment of the present disclosure, a tip of the bronchoscope of the bronchoscope path display device has a tip positioning device. The tip positioning device is for generating the tip positioning signal and transmitting the tip positioning signal to the processing device.

In one embodiment of the present disclosure, the tip positioning device is selected from the group consisting of accelerometer and gyroscope.

In the bronchoscope path assistance system of the present disclosure, by generating a composite image signal including an assistance positioning information, a user can be effectively assisted for a bronchoscope to correctly enter a desired lung area, so as to reduce risks resulted on a patient or an inspection subject and enhance efficiency of treatment, inspection or sampling. In the positioning mouthpiece of the present disclosure, with the mouthpiece and the positioning device provided, a relative movement between the bronchoscope and the mouthpiece can be precisely detected. In the bronchoscope path display device of the present disclosure, by receiving a positioning signal, a composite image signal including an assistance positioning information can be generated, and a user can be effectively assisted for a bronchoscope to correctly enter a desired lung area, so as to reduce risks resulted on a patient or an inspection subject and enhance efficiency of treatment, inspection or sampling.

### BRIEF DESCRIPTION OF THE DRAWINGS

For better clear illustration, thicknesses or sizes of the layers depicted in the drawings may be emphasized, omitted or drawn in brief. Meanwhile, the sizes of the elements do not reflected actual sizes thereof.

FIG. 1 is a schematic diagram of a bronchoscope path assistance system according to a first embodiment of the present disclosure.

FIG. 2 is a schematic diagram of a bronchoscope path assistance system in a state of use according to the first embodiment of the present disclosure.

FIG. 3 is a schematic diagram of a bronchoscope path assistance system in another state of use according to the first embodiment of the present disclosure.

FIG. 4 is a schematic diagram of a bronchoscope path assistance system in yet another state of use according to the first embodiment of the present disclosure.

FIG. 5 is a schematic diagram of a positioning mouthpiece according to a second embodiment of the present disclosure.

FIG. 6 is a schematic diagram of a positioning mouthpiece according to a third embodiment of the present disclosure.

FIG. 7 is a schematic diagram of a positioning mouthpiece from another angle of view according to the third embodiment of the present disclosure.

FIG. 8 is a schematic diagram of a positioning mouthpiece according to a fourth embodiment of the present disclosure.

FIG. 9 is a schematic diagram of a positioning mouthpiece from another angle of view according to the fourth embodiment of the present disclosure.

FIG. 10 is a schematic diagram of a bronchoscope path display device according to a fifth embodiment of the present disclosure.

### DETAILED DESCRIPTION OF THE INVENTION

To facilitate understanding of the object, characteristics and effects of this present disclosure, embodiments together with the attached drawings for the detailed description of the present disclosure are provided.

### First Embodiment

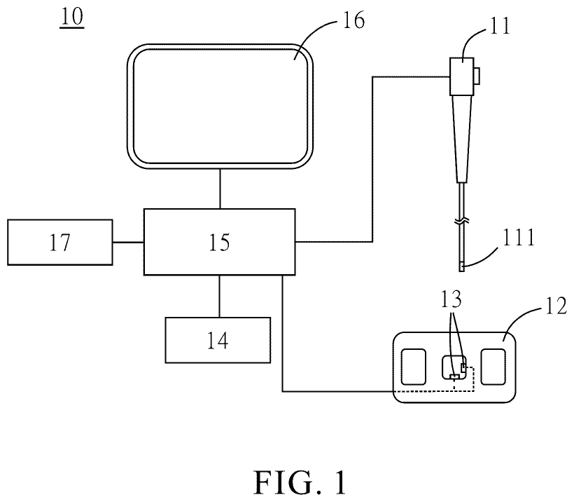

A bronchoscope path assistance system according to a first embodiment of the present disclosure is as shown in FIG. 1.

The bronchoscope path assistance system 10 according to the first embodiment of the present disclosure includes a bronchoscope 11, a mouthpiece 12, a positioning device 13, a memory device 14, a processing device 15, a display unit 16 and an input unit 17.

The bronchoscope 11 has a camera device 111 on one end thereof, and is for generating an image signal.

The mouthpiece 12 has an opening at a center thereof, and is adapted to guide the bronchoscope 11 to pass through a mouth of an inspection subject via the opening. In a preferred embodiment, the mouthpiece 12 may be fixed on a patient or an inspection subject by a hook-and-eye fastener or a fastening string; however, the present application is not limited to the examples above.

The positioning device 13 is disposed on the mouthpiece 12, and is for detecting a relative movement between the bronchoscope 11 and the mouthpiece 12 to issue a positioning signal. More specifically, the positioning signal may include information such as a distance by which the bronchoscope 11 enters deep into the lungs from the mouthpiece 12, an axial initial orientation of the bronchoscope 11 while passing through the mouthpiece 12, and an axial rotation magnitude of the bronchoscope 11 from the initial orientation; however, the present application is not limited to the examples above.

In this embodiment of the present disclosure, the positioning device 13 includes two positioning cameras, the bronchoscope 11 has a plurality of equidistant positioning marks, and the positioning device 13 detects the relative movement between the bronchoscope 11 and the mouthpiece 12 according to images of the positioning marks captured by the two positioning cameras; however, the present disclosure is not limited to the examples above. In a preferred embodiment, the positioning device may include four positioning cameras, which are located on upper, lower, left and right sides of the opening of the mouthpiece, respectively. In another embodiment of the present disclosure, the positioning device may include a plurality of positioning rollers. During use, the plurality of positioning rollers are in contact with the bronchoscope, and the positioning device detects the relative movement between the bronchoscope and the mouthpiece according to rotation magnitudes of the plurality of positioning rollers. In another preferred embodiment, the positioning device may include four positioning rollers, which are located on upper, lower, left and right sides of the opening of the mouthpiece, respectively.

The memory device 14 is for storing path information associated with branch paths of bronchi. For example, the memory device 154 may be a mechanical hard drive, a solid-state hard drive, a flash memory device, a memory card or an optical disc; however, the present application is not limited to the examples above. More specifically, the path information may include branches of the human body from the trachea to individual ends of the lungs, relative orientations among individual paths, and relative distances among individual branch points.

The processing device 15 is for receiving the image signal and the positioning signal and accessing the path information to generate a composite image signal according to the image signal, the positioning signal and the path information, wherein the composite image signal includes the image signal and an assistance positioning information generated according to the positioning signal and the path information. For example, the processing device 15 may be a desktop computer, a laptop computer, a tablet computer or a smartphone; however, the disclosure is not limited to the examples above.

The display unit 16 is for receiving and displaying the composite image signal. For example, the display unit 16 may be a liquid crystal display (LCD), an organic light emitting diode (OLED) display or a plasma display; however, that the present application is not limited to the examples above.

The input unit 17 is for receiving a determined path information input by a user. For example, the input unit 17 may be a keyboard, a mouse, or be integrated with the display unit 16 into one touch screen; however, the present application is not limited to the examples above.

After the user inputs the determined path information, the processing device 15 generates a new assistance positioning information according to the determined path information, the positioning signal and the path information.

In a preferred embodiment of the present disclosure, the memory device 14, the processing device 15, a display unit 16 and the input unit 17 are integrated into a tablet computer.

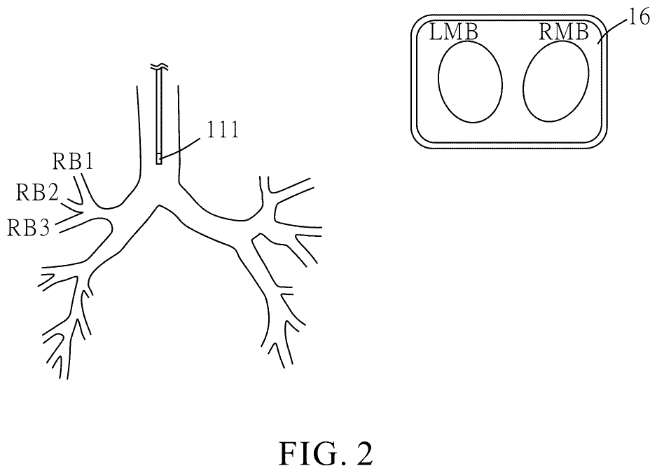

FIG. 2 to FIG. 4 show a series of schematic diagrams of the bronchoscope path assistance system in a state of use according to the first embodiment of the present disclosure. The left shows a position of the camera device 111 of the bronchoscope 11 in the lungs, and the right shows the composite image signal displayed on the display unit 16. In the state of use shown in FIG. 2 to FIG. 4, a user wishes to place the bronchoscope 11 into a right apical bronchus.

When the camera device 111 of the bronchoscope 11 is located at the position as shown in FIG. 2, the processing device 15 may determine, according to the positioning signal transmitted from the positioning device 13 and the path information stored in the memory device 14, that the camera device 111 is located near a junction between the right main bronchus and the left main bronchus.

Thus, the processing device 15 may further integrate the image signal generated by the camera device 111 of the bronchoscope 11, and generate, according to path information associated with relative orientations of the right main bronchus and the left main bronchus stored in the memory device 14, the composite image signal displayed on the display unit 16 as shown in FIG. 2, for the assistance positioning information to be displayed at a part of the corresponding bronchus.

The ellipsoid on the right in the composite signal displayed on the display unit 16 in FIG. 2 is the opening of the right main bronchus captured by the camera device 111, and the ellipsoid on the left is the opening of the left main bronchus captured by the camera device 111. The composite image signal further includes corresponding assistance positioning information LMB and RMB generated according to the positioning signal and the path information, wherein the assistance positioning information RMB represents that the right main bronchus is displayed on the upper right of the opening of the corresponding right main bronchus, and LMB represents that the left main bronchus is displayed on the upper left of the opening of the corresponding left main bronchus. Thus, by using the assistance positioning information in the composite image signal, a user is able to guide the bronchoscope 11 to enter the right main bronchus on the right and to further enter the right apical bronchus instead of incorrectly entering the left main bronchus on the left.

In a preferred embodiment, the positioning signal transmitted by the positioning device 13 includes information such as an axial initial orientation of the bronchoscope 11 while passing through the mouthpiece 12 and the axial rotation magnitude of the bronchoscope 11. Thus, even if the bronchoscope 11 passes through the mouthpiece 12 by a different initial orientation or rotates axially during the operation process, the processing device 15 is still able to adjust the assistance positioning information according to the positioning signal, so as to ensure that the assistance positioning information is maintained at the position of the opening of a corresponding bronchus.

For example, assume that the bronchoscope 11 rotates axially by 180° relative to the state shown in FIG. 2 in a way that the opening of the right main bronchus is located on the left and the left main bronchus is located on the right. At this point, the processing device 15 may adjust the positions of the assistance positioning information LMB and RMB in the composite signal according to the information of the rotation magnitude of the bronchoscope 11, so that the assistance positioning information LMB is located at a position on the left near the opening of the right main bronchus and the assistance positioning information RMB is located at a position on the right near the opening of the left main bronchus.

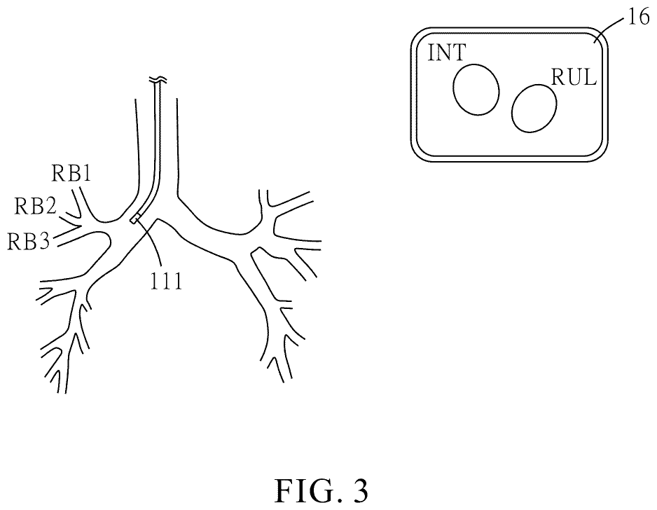

When the camera device 111 of the bronchoscope 11 is located at the position as shown in FIG. 3, the user inputs a determined path information representing entering the right main bronchus. At this point, the processing device 15 may determine, according to the determined path information, the positioning signal transmitted by the positioning device 13 and the path information stored in the memory device 14, that the camera device 111 is located near a junction between the intermediate bronchus and the right upper lobe bronchus.

Thus, the processing device 15 may further integrate the image signal generated by the camera device 111 of the bronchoscope 11, and generate, according to the path information associated with relative orientations of the intermediate bronchus and the right upper lobe bronchus stored in the memory device 14, the composite image signal displayed on the display unit 16 as shown in FIG. 3, for the assistance positioning information to be displayed at a part of the corresponding bronchus.

The ellipsoid on the lower right in the composite signal displayed on the display unit 16 in FIG. 3 is the opening of the right upper lobe bronchus captured by the camera device 111, and the ellipsoid on the upper left is the opening of the intermediate bronchus captured by the camera device 111. The composite image signal further includes corresponding assistance positioning information INT and RUL generated according to the positioning signal and the path information, wherein the assistance positioning information INT represents that the intermediate bronchus is displayed on the upper left of the opening of the corresponding intermediate bronchus, and RUL represents that the right upper lobe bronchus is displayed on the upper right of the opening of the corresponding right upper lobe bronchus. Thus, by using the assistance positioning information in the composite image signal, a user is able to guide the bronchoscope 11 to enter the right upper lobe bronchus on the lower right and to further enter the right apical bronchus instead of incorrectly entering the intermediate bronchus on the upper left.

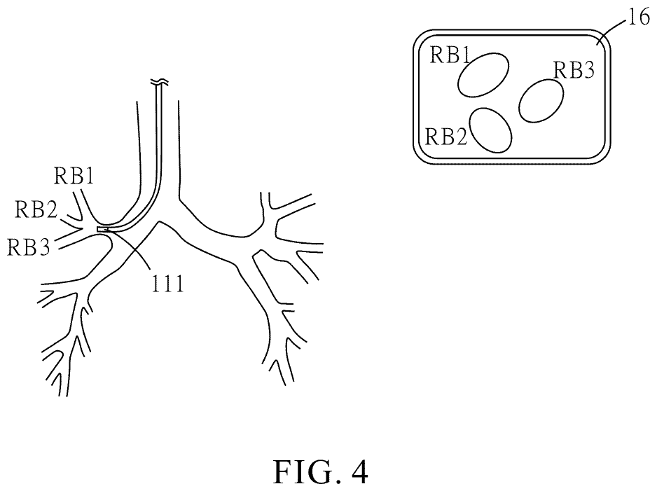

When the camera device 111 of the bronchoscope 11 is located at the position as shown in FIG. 4, the user inputs a determined path information representing entering the right upper lobe bronchus. At this point, the processing device 15 may determine, according to the determined path information, the positioning signal transmitted by the positioning device 13 and the path information stored in the memory device 14, that the camera device 111 is located near a junction of the right apical bronchus, the right posterior bronchus and right anterior bronchus.

Thus, the processing device 15 may further integrate the image signal generated by the camera device 111 of the bronchoscope 11, and generate, according to the path information associated with relative orientations of the right apical bronchus, the right posterior bronchus and right anterior bronchus stored in the memory device 14, the composite image signal displayed on the display unit 16 as shown in FIG. 4, for the assistance positioning information to be displayed at a part of the corresponding bronchus.

The ellipsoid on the upper left in the composite signal displayed on the display unit 16 in FIG. 4 is the opening of the right apical bronchus captured by the camera device 111, the ellipsoid on the lower left is the opening of the right posterior bronchus captured by the camera device 111, and the ellipsoid on the right is the opening of the right anterior bronchus captured by the camera device 111. The composite image signal further includes corresponding assistance positioning information RB1, RB2 and RB3 generated according to the positioning signal and the path information, wherein the assistance positioning information RB1 represents that the right apical bronchus is displayed on the upper left of the opening of the corresponding right apical bronchus, the assistance positioning information RB2 represents that the right posterior bronchus is displayed on the left of the opening of the corresponding right posterior bronchus, and the assistance positioning information RB3 represents that the right anterior bronchus is displayed on the upper right of the opening of the corresponding right anterior bronchus. Thus, by using the assistance positioning information in the composite image signal, a user is able to guide the bronchoscope 11 to enter the right apical bronchus on the upper left instead of incorrectly entering the right posterior bronchus on the lower left or incorrectly entering the right anterior bronchus on the right.

Once the user has completely performed treatment, inspection or sampling for the right apical bronchus, the user may slightly extract the bronchoscope 11 to return the bronchoscope 11 to the position as shown in FIG. 3. At this point, the processing device 15 may determine, according to the determined path information, the positioning signal transmitted by the positioning device 13 and the path information stored in the memory device 14, that the camera device 111 has returned to the junction between the intermediate bronchus and the right upper lobe bronchus. Thus, the processing device 15 may again integrate the image signal generated by the camera device 111 of the bronchoscope 11, and again generate the composite image signal displayed on the display unit 16 as shown in FIG. 3. At this point, by using the assistance positioning information of the composite signal, the user is able to guide the bronchoscope 11 to alternatively enter the intermediate bronchus but not the right upper lobe bronchus, so as to further perform treatment, inspection or sampling of the intermediate bronchus and bronchi at the downstream thereof.

When the bronchoscope 11 alternatively enter the intermediate bronchus, the user inputs a determined path information representing entering the intermediate bronchus. At this point, the processing device 15 may display the composite image signal of the downstream of the intermediate bronchus according to the determined path information, the positioning signal transmitted by the positioning device 13 and the path information stored in the memory device 14. Thus, by using the assistance positioning information of the composite signal, the user is able to guide the bronchoscope 11 to enter a bronchus at the downstream of the intermediate bronchus.

By repeating the steps above, by using the assistance positioning information of the composite signal, the user is able to guide the bronchoscope 11 to enter any desired bronchus of the lungs, so as to perform treatment, inspection or sampling on the desired bronchus.

As described above, with the bronchoscope path assistance system of the present disclosure, by generating a composite image signal including an assistance positioning information, a user can be effectively assisted for a bronchoscope to correctly enter a desired lung area, so as to reduce risks resulted on a patient or an inspection subject and enhance efficiency of treatment, inspection or sampling.

In a preferred embodiment, a tip of the bronchoscope included in the bronchoscope path assistance system has a snake bone structure. With the configuration of the snake bone structure, it is ensured that the tip of the bronchoscope has sufficient rigidity and bendability to be readily enter deep into the lungs; however, the present application is not limited to the example above. Moreover, in a more preferred embodiment, the bronchoscope has a distal bending mechanism, the user may bend the tip of the bronchoscope by the distal bending mechanism so as to place the bronchoscope deep into the lungs, and the bronchoscope further has a sensing mechanism to sense a bending state of the tip of the bronchoscope. In this more preferred embodiment, the bronchoscope path assistance system further refers to the bending state of the tip of the bronchoscope sensed by the sensing mechanism to adjust a display position of the assistance positioning information in the composite signal, so as to enable the user to obtain more precise positioning information; however, the present application is not limited to the example above.

In another preferred embodiment, the memory device included in the bronchoscope path assistance system further stores image data associated with branch paths of bronchi. In this more preferred embodiment, the bronchoscope path assistance system further compares and analyzes, by artificial intelligence, the image signal generated by the bronchoscope with the image data stored in the memory device, and adjusts the display position of the assistance positioning information in the composite image signal for the user to obtain more precise positioning information; however, the present application is not limited to the examples above.

In a preferred embodiment of the present disclosure, the tip of the bronchoscope included in the bronchoscope path assistance system has a tip positioning device. The tip positioning device is for generating a tip positioning signal, and transmitting the tip positioning signal to the processing device, wherein the processing device generates the composite image signal further according to the tip positioning signal; however, the present application is not limited to the examples above. In a more preferred embodiment of the present disclosure, the tip positioning device is selected from the group consisting of accelerometer and gyroscope; however, the present application is not limited to the examples above. For example, the tip positioning device may include three accelerometers and three gyroscopes to provide more precise positioning information; however, the present application is not limited to the examples above.

In the embodiment including the accelerometers, a displacement of the tip of the bronchoscope relative to a direction of the gravitational force may be determined by the accelerometers. In this more preferred embodiment, the bronchoscope path assistance system further refers to the displacement of the tip of the bronchoscope sensed by the accelerometers to adjust the display position of the assistance positioning information in the composite signal, so as to prevent misjudgment caused by different postures of the user (for example, reclining on the back or reclining sideways) for the user to obtain more precise positioning information; however, the present application is not limited to the example above.

In the embodiment including the gyroscopes, an axial change generated of the tip of the bronchoscope caused by bending or turning of passages of a bronchus may be detected by the gyroscopes. In this more preferred embodiment, the bronchoscope path assistance system further refers to the axial change of the tip of the bronchoscope sensed by the gyroscopes to adjust the display position of the assistance positioning information in the composite signal, so as to prevent misjudgment caused by bending and turning of the passages of the bronchus for a user to obtain more precise positioning information; however, the present application is not limited to the examples above.

In a preferred embodiment of the present disclosure, the mouthpiece included in the bronchoscope path assistance system has a plurality of initial orientation positioning marks located near the opening of the mouthpiece. When the camera device on one end of the bronchoscope is about to pass through the mouthpiece, the image signal generated by the camera device is to include the initial orientation positioning marks, and the processing device further analyzes the image signal including the plurality of initial orientation positioning marks to determine an initial orientation and generates the composite image signal further according to the initial orientation; however, the present application is not limited to the examples above. In this preferred embodiment, by configuring the mouthpiece to have the plurality of initial orientation positioning marks, the axial initial orientation while the bronchoscope passing through the mouthpiece can be more precisely determined. Thus, even if the initial orientation while the bronchoscope passes through the mouthpiece may differ due to the posture of the inspection subject or habits of the user, the composite image signal generated by the bronchoscope path assistance system is still able to perform corresponding corrections to provide the user with precise positioning information.

### Second Embodiment

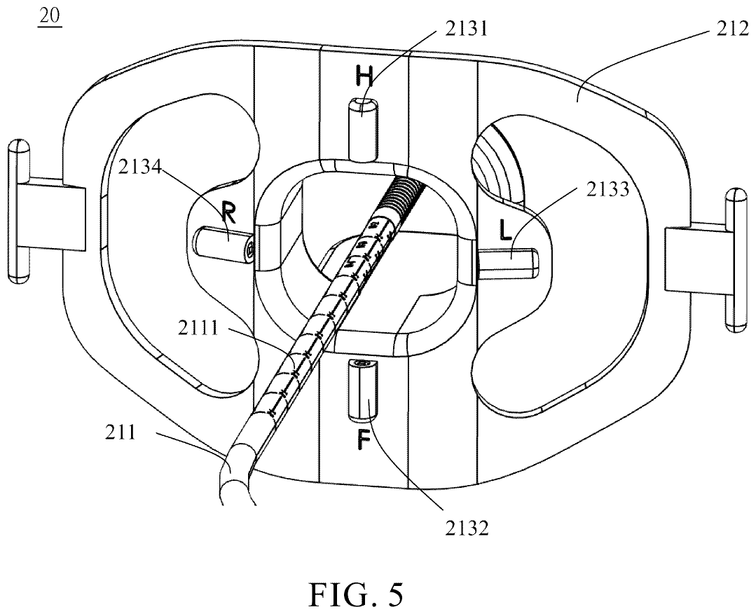

As shown in FIG. 5, a positioning mouthpiece 20 of the second embodiment includes: a mouthpiece 212, having an opening at a center thereof, adapted to guide a bronchoscope 211 to pass through the mouth of an inspection subject via the opening; and a positioning device, disposed on the mouthpiece 212, for detecting a relative movement between the bronchoscope 211 and the mouthpiece 212 to issue a positioning signal.

The positioning device disposed on the mouthpiece 212 of the positioning mouthpiece 20 of the second embodiment includes four positioning cameras 2131, 2132, 2133 and 2134, which are located on upper, lower, left and right sides of the opening of the mouthpiece 212, respectively.

For example, the positioning mouthpiece 20 of the second embodiment may be used to guide the bronchoscope 211 having a plurality of equidistant positioning marks 2111 as shown in FIG. 5, and the positioning device detects the relative movement between the bronchoscope 211 and the mouthpiece 212 according to images of the positioning marks captured by he four positioning cameras 2131, 2132, 2133 and 2134; however, the present application is not limited to the examples above. Thus, the positioning mouthpiece 20 of the second embodiment is able to more precisely detect the relative movement between the bronchoscope 211 and the mouthpiece 212.

The positioning mouthpiece 20 of the second embodiment is applicable to the bronchoscope path assistance system described in the first embodiment, so as to enhance the accuracy of the composite image signal including the assistance positioning information generated by the bronchoscope path assistance system.

The mouthpiece 212 included in the positioning mouthpiece 20 of the second embodiment has a plurality of initial orientation positioning marks H, F, R and L, which are located near the opening of the mouthpiece 212; however, the present application is not limited to the examples above. When the positioning mouthpiece 20 of the second embodiment is applied in the bronchoscope path assistance system described in the first embodiment, the positioning mouthpiece 20 is fixed at the mouth of an inspection subject in an particular orientation, such that the four initial orientation positioning marks H, F, R and L are located on orientations corresponding to the head (H), the foot (F), the right hand (R) and the left hand (L) of the inspection subject. When a camera device on one end of the bronchoscope 211 is about to pass through the mouthpiece 212, an image signal generated by the camera device is to include the initial orientation positioning marks H, F, R and L. At this point, the processing device in the bronchoscope path assistance system described in the first embodiment may further analyze the image signal including the plurality of initial orientation positioning marks to determine an initial orientation, and generate the composite image signal further according to the initial orientation.

By configuring the mouthpiece 212 of the positioning mouthpiece 20 of the second embodiment to have the plurality of orientation positioning marks, when the positioning mouthpiece 20 is applied in the bronchoscope path assistance system described in the first embodiment, the axial initial orientation while the bronchoscope passing through the mouthpiece can be more precisely determined.

### Third Embodiment

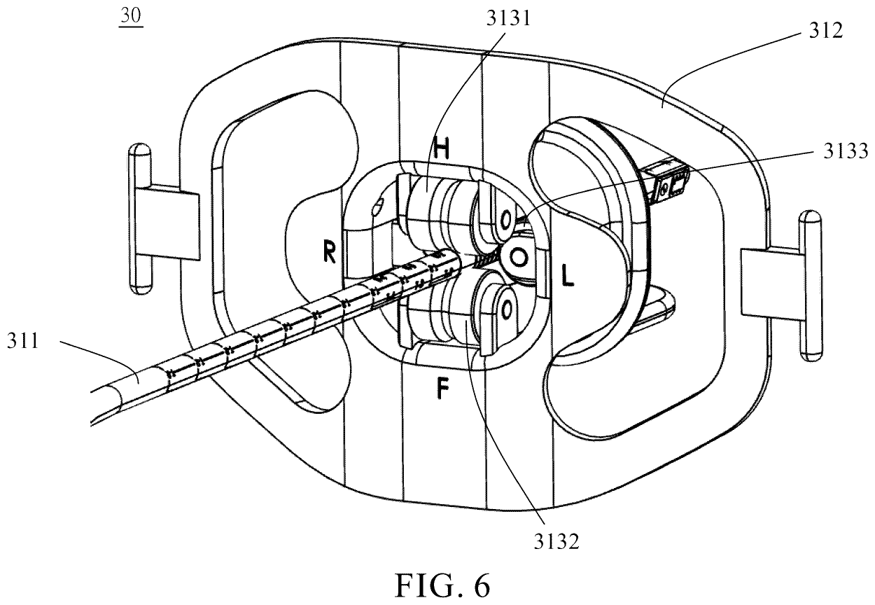

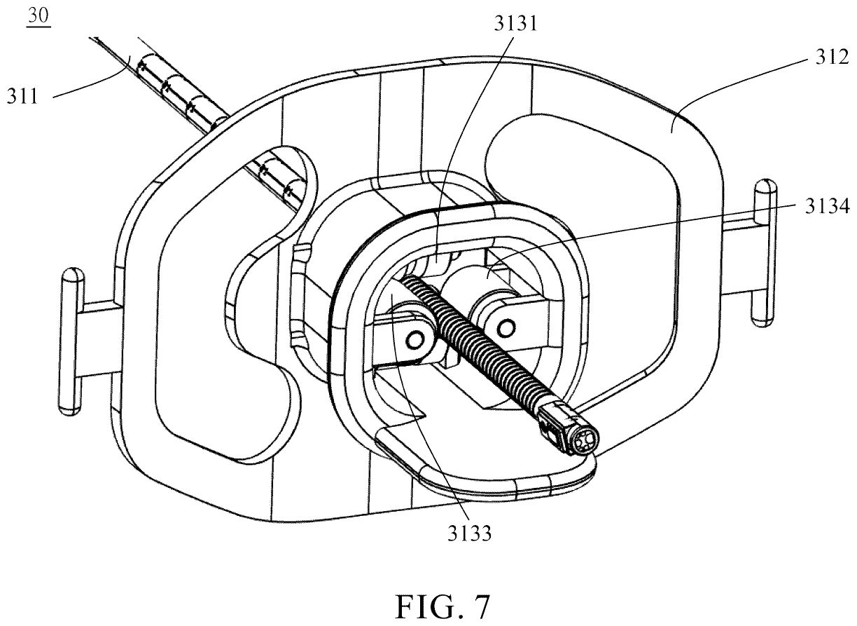

As shown in FIG. 6 and FIG. 7, a positioning mouthpiece 30 of the third embodiment includes: a mouthpiece 312, having an opening at a center thereof, adapted to guide a bronchoscope 311 to pass through the mouth of an inspection subject via the opening; and a positioning device, disposed on the mouthpiece 312, for detecting a relative movement between the bronchoscope 311 and the mouthpiece 312 to issue a positioning signal.

The positioning device disposed on the mouthpiece 312 of the positioning mouthpiece 30 of the third embodiment includes four positioning rollers 3131, 3132, 3133 and 3134, which are located on upper, lower, left and right sides of the opening of the mouthpiece 312, respectively. The positioning rollers 3131 and 3132 clamp the bronchoscope 311 in a top-down manner, and directions of axes of the positioning rollers 3131 and 3132 are perpendicular to a direction of an axis of the bronchoscope 311, so as to detect forward and backward movements between the bronchoscope 311 and the mouthpiece 312 according to rotation magnitudes of the positioning rollers 3131 and 3132. The positioning rollers 3133 and 3134 clamp the bronchoscope 311 in a left-right manner, and directions of axes of the positioning rollers 3133 and 3134 are parallel to the direction of the axis of the bronchoscope 311, so as to detect rotations between the bronchoscope 311 and the mouthpiece 312 according to rotation magnitudes of the positioning rollers 3133 and 3134. Thus, the positioning mouthpiece 30 of the third embodiment is able to more precisely detect the relative movement between the bronchoscope 311 and the mouthpiece 312.

Similar to the second embodiment, the mouthpiece 312 included in the positioning mouthpiece 30 of the third embodiment has a plurality of initial orientation positioning marks H, F, R and L, which are located near the opening of the mouthpiece 312; however, the present application is not limited to the examples above. Applications details and functions of the initial orientation positioning marks H, F, R and L of the third embodiment are the same as those of the second embodiment, and are omitted herein.

### Fourth Embodiment

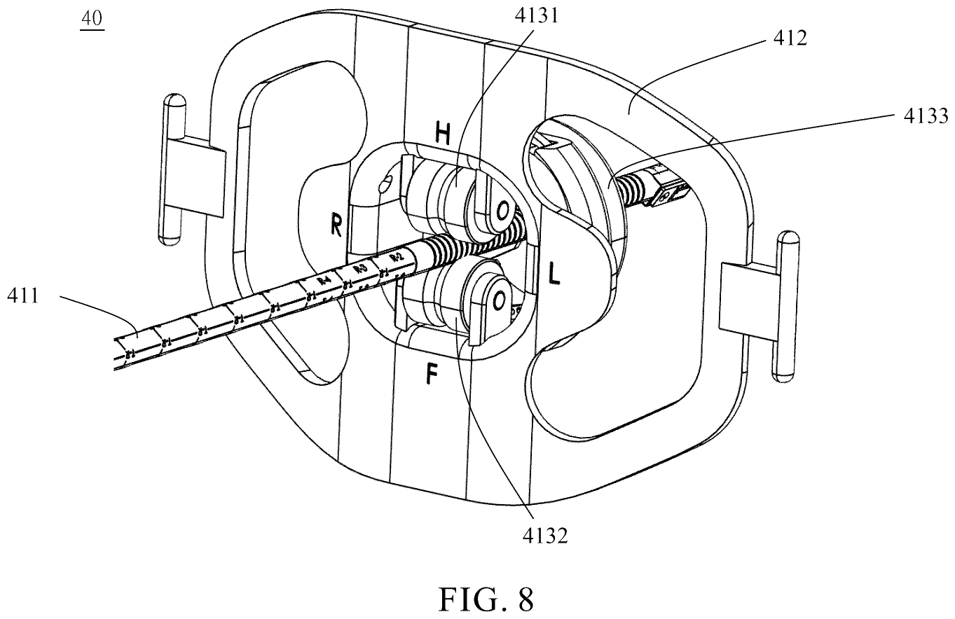

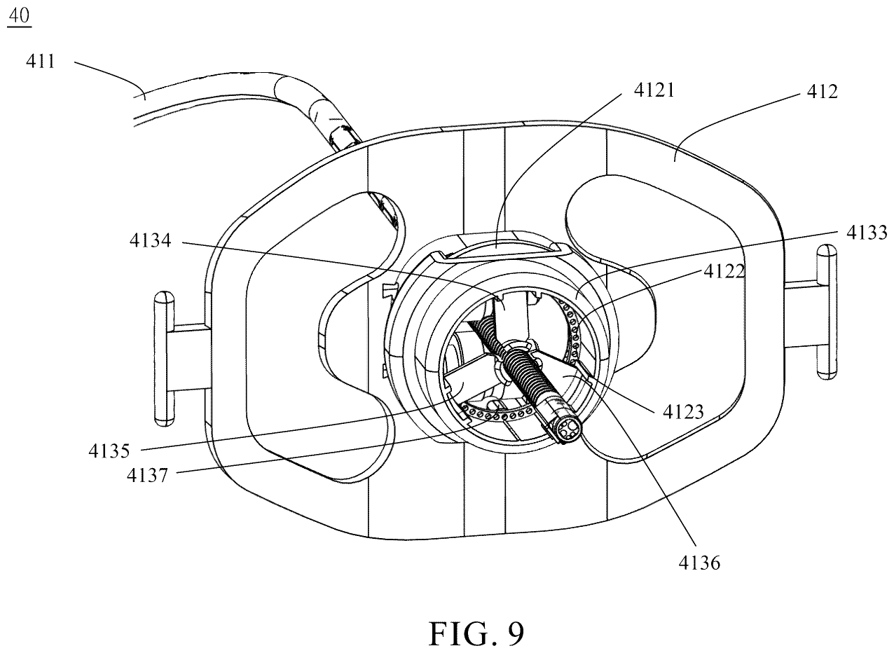

As shown in FIG. 8 and FIG. 9, a positioning mouthpiece 40 of the fourth embodiment includes: a mouthpiece 412, having an opening at a center thereof, adapted to guide a bronchoscope 411 to pass through the mouth of an inspection subject via the opening; and a positioning device, disposed on the mouthpiece 412, for detecting a relative movement between the bronchoscope 411 and the mouthpiece 412 to issue a positioning signal.

The positioning device disposed on the mouthpiece 412 of the positioning mouthpiece 40 of the fourth embodiment includes two positioning rollers 4131 and 4132, which are located on upper and lower sides of the opening of the mouthpiece 412, respectively. The positioning rollers 4131 and 4132 clamp the bronchoscope 411 in a top-down manner, and directions of axes of the positioning rollers 4131 and 4132 are perpendicular to a direction of an axis of the bronchoscope 411, so as to detect front and back movements between the bronchoscope 411 and the mouthpiece 412 according to rotation magnitudes of the positioning rollers 4131 and 4132. The positioning device disposed on the mouthpiece 412 of the positioning mouthpiece 40 of the fourth embodiment includes a positioning rotor 4133, which is disposed on a guide rail 4121 located on the mouthpiece 412. The positioning rotor 4133 and the guide rail 4121 are disposed around the opening of the mouthpiece 412, and the positioning rotor 4133 has a plurality of holes 4122 and 4123 in an annular arrangement. The positioning rotor 4133 is provided with a plurality of clamping devices 4134, 4135 and 4136, which are in an interference fit with the bronchoscope 411, and a direction of an axis of the positioning rotor 4133 overlaps a direction of an axis of the bronchoscope 411. Thus, when the bronchoscope 411 rotates, the positioning rotor 4133 is driven to perform a rotation of the same magnitude. The positioning device disposed on the mouthpiece 412 in the bronchoscope path assistance system of the fourth embodiment further includes an encoder 4137. The encoder 4137 is disposed at a position relative to the plurality of holes 4122 and 4123 on the positioning rotor 4133, and detects a rotation direction and a rotation angle between the bronchoscope 411 and the mouthpiece 412 according to directions and magnitudes of displacements of the plurality of holes 4122 and 4123. Thus, the positioning mouthpiece 40 of the fourth embodiment is able to more precisely detect the relative movement between the bronchoscope and the mouthpiece.

Similar to the second embodiment, the mouthpiece 412 included in the positioning mouthpiece 40 of the fourth embodiment has a plurality of initial orientation positioning marks H, F, R and L, which are located near the opening of the mouthpiece 412; however, the present application is not limited to the examples above. Applications details and functions of the initial orientation positioning marks H, F, R and L of the fourth embodiment are the same as those of the second embodiment, and are omitted herein.

### Fifth Embodiment

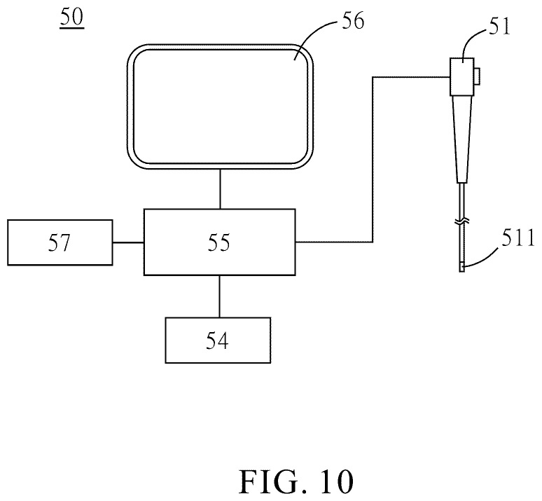

As shown in FIG. 10, a bronchoscope path display device 50 of the fifth embodiment includes: a bronchoscope 51, having a camera device 511 on one end thereof, for generating an image signal; a memory device 54, for storing path information associated with branch paths of bronchi; a processing device 55, for receiving the image signal and a positioning signal and accessing the path information to generate a composite image signal according to the image signal, the positioning signal and the path information, wherein the composite image signal includes the image signal and an assistance positioning information generated according to the positioning signal and the path information; a display unit 56, for receiving and displaying the composite image signal; and an input unit 57, for receiving a determined path information input by a user, wherein after the user inputs the determined path information, the processing device 55 generates a new assistance positioning information according to the determined path information, the positioning signal and the path information.

In the bronchoscope path display device 50 of the fifth embodiment, by receiving a positioning signal, a composite image signal including an assistance positioning information can be generated, and a user can be effectively assisted for a bronchoscope to correctly enter a desired lung area, so as to reduce risks resulted on a patient or an inspection subject and enhance efficiency of treatment, inspection or sampling.

For example, the bronchoscope path display device 50 of the fifth embodiment may receive the positioning signals generated by the positioning mouthpieces 20, 30 and 50 as described in the second to fourth embodiments, so as to generate the composite image signal including the assistance positioning information; however, the present application is not limited to the examples above.

In a preferred embodiment of the present disclosure, a tip of the bronchoscope of the bronchoscope path display device has a tip positioning device. The tip positioning device is for generating the positioning signal and transmitting the positioning signal to the processing device; however, the present application is not limited to the examples above. In a more preferred embodiment of the present disclosure, the tip positioning device is selected from the group consisting of accelerometer and gyroscope; however, the present application is not limited to the examples above. For example, the tip positioning device may include three accelerometers and three gyroscopes to provide more precise positioning information; however, the present application is not limited to the examples above.

In the embodiment including the accelerometers, a displacement of the tip of the bronchoscope relative to a direction of the gravitational force may be determined by the accelerometers to generate the positioning signal. Thus, misjudgment caused by different postures of the user (for example, reclining on the back or reclining sideways) can be prevented for a user to obtain more precise positioning information; however, the present application is not limited to the example above.

In the embodiment including the gyroscopes, an axial change generated of the tip of the bronchoscope caused by bending or turning of passages of the bronchi may be detected by the gyroscopes to generate the positioning signal. Thus, misjudgment caused by bending and turning of the passages of the bronchi can be prevented for a user to obtain more precise positioning information; however, the present application is not limited to the examples above.

The present invention is described by way of the preferred embodiments above. A person skilled in the art should understand that, these embodiments are merely for describing the present invention and are not to be construed as limitations to the scope of the present invention. It should be noted that all equivalent changes, replacements and substitutions made to the embodiments are to be encompassed within the scope of the present invention. Therefore, the scope of protection of the present invention should be accorded with the broadest interpretation of the appended claims.

While the present disclosure has been described by means of specific embodiments, numerous modifications and variations could be made thereto by those skilled in the art without departing from the scope and spirit of the present disclosure set forth in the claims.

What is claimed is:

1. A bronchoscope path assistance system, comprising:
a bronchoscope, having a camera device on one end thereof, for generating an image signal;
a mouthpiece, having an opening at a center thereof, adapted to guide the bronchoscope to pass through a mouth of an inspection subject via the opening;
a positioning device, disposed on the mouthpiece, for detecting a relative movement between the bronchoscope and the mouthpiece to issue a positioning signal;
a memory device, for storing path information associated with branch paths of bronchi;
a processing device, for receiving the image signal and the positioning signal and accessing the path information to generate a composite image signal according to the image signal, the positioning signal and the path information, wherein the composite image signal comprises the image signal and an assistance positioning information generated according to the positioning signal and the path information;
a display unit, for receiving and displaying the composite image signal; and
an input unit, for receiving a determined path information input by a user,
wherein, after the user inputs the determined path information, the processing device generates a new assistance positioning information according to the determined path information, the positioning signal and the path information.

2. The bronchoscope path assistance system according to claim 1, wherein the positioning device comprises a plurality of positioning cameras, the bronchoscope has a plurality of equidistant positioning marks, and the positioning device detects the relative movement between the bronchoscope and the mouthpiece according to images of the positioning marks captured by the plurality of positioning cameras.

3. The bronchoscope path assistance system according to claim 2, wherein the positioning device comprises four positioning cameras, which are located on upper, lower, left and right sides of the opening of the mouthpiece, respectively.

4. The bronchoscope path assistance system according to claim 1, wherein the positioning device comprises a plurality of positioning rollers, and during use, the plurality of positioning rollers are in contact with the bronchoscope, and the positioning device detects the relative movement between the bronchoscope and the mouthpiece according to rotation magnitudes of the plurality of positioning rollers.

5. The bronchoscope path assistance system according to claim 4, wherein the positioning device comprises four positioning rollers, which are located on upper, lower, left and right sides of the opening of the mouthpiece, respectively.

6. The bronchoscope path assistance system according to claim 4, wherein the positioning device further comprises:
a positioning rotor, disposed on a guide rail on the mouthpiece, wherein the positioning rotor and the guide rail are disposed around the opening of the mouthpiece, and the positioning rotor has thereon a plurality of holes in an annular arrangement and is provided with a plurality of elastic clamping devices in an interference fit with the bronchoscope; and
an encoder, disposed at a position relative to the plurality of holes on the positioning rotor, and detecting a rotation direction and a rotation angle between the bronchoscope and the mouthpiece according to a direction and an magnitude of a displacement of the plurality of holes.

7. The bronchoscope path assistance system according to claim 1, wherein the display unit and the input unit are integrated into a touch screen.

8. The bronchoscope path assistance system according to claim 1, wherein the memory device, the processing device, the display unit and the input unit are integrated into a tablet computer.

9. The bronchoscope path assistance system according to claim 1, wherein a tip of the bronchoscope has a tip positioning device, and the tip positioning device is for generating a tip positioning signal and transmitting the tip positioning signal to the processing device, wherein the processing device generates the composite image signal further according to the tip positioning signal.

10. The bronchoscope path assistance system according to claim 9, wherein the tip positioning device is selected from the group consisting of accelerometer and a gyroscope.

11. The bronchoscope path assistance system according to claim 1, wherein the mouthpiece has a plurality of initial orientation positioning marks located near the opening of the mouthpiece; wherein when the camera device on one end of the bronchoscope is about to pass through the mouthpiece, the image signal generated by the camera device is to include the initial orientation positioning marks, and the processing device further analyzes the image signal including the plurality of initial orientation positioning marks to determine an initial orientation and generates the composite image signal further according to the initial orientation.

12. A positioning mouthpiece, comprising:
a mouthpiece, having an opening at a center thereof, adapted to guide a bronchoscope to pass through a mouth of an inspection subject via the opening; and
a positioning device, disposed on the mouthpiece, for detecting a relative movement between the bronchoscope and the mouthpiece to issue a positioning signal.

13. The positioning mouthpiece according to claim 12, wherein the positioning device comprises four positioning cameras, which are located on upper, lower, left and right sides of the opening of the mouthpiece, respectively.

14. The positioning mouthpiece according to claim 12, wherein the positioning device comprises a plurality of positioning rollers, and during use, the plurality of positioning rollers are in contact with the bronchoscope, and the positioning device detects the relative movement between the bronchoscope and the mouthpiece according to rotation magnitudes of the plurality of positioning rollers.

15. The positioning mouthpiece according to claim 14, wherein the positioning device comprises four positioning rollers, which are located on upper, lower, left and right sides of the opening of the mouthpiece, respectively.

16. The positioning mouthpiece according to claim 14, wherein the positioning device of the positioning mouthpiece further comprises:
a positioning rotor, disposed on a guide rail on the mouthpiece, wherein the positioning rotor and the guide rail are disposed around the opening of the mouthpiece, and the positioning rotor has thereon a plurality of holes in an annular arrangement and is provided with a plurality of elastic clamping devices in an interference fit with the bronchoscope; and
an encoder, disposed at a position relative to the plurality of holes on the positioning rotor, and detecting a rotation direction and a rotation angle between the bronchoscope and the mouthpiece according to a direction and an magnitude of a displacement of the plurality of holes.

17. The positioning mouthpiece according to claim 12, wherein the mouthpiece has a plurality of initial orientation positioning marks located near the opening of the mouthpiece.

18. A bronchoscope path display device, comprising:
a bronchoscope, having a camera device on one end thereof, for generating an image signal;
a memory device, for storing path information associated with branch paths of bronchi;
a processing device, for receiving the image signal and a positioning signal and accessing the path information to generate a composite image signal according to the image signal, the positioning signal and the path information, wherein the composite image signal includes the image signal and an assistance positioning information generated according to the positioning signal and the path information;
a display unit, for receiving and displaying the composite image signal; and
an input unit, for receiving a determined path information input by a user,
wherein, after the user inputs the determined path information, the processing device generates a new assistance positioning information according to the determined path information, the positioning signal and the path information.

19. The bronchoscope path display device according to claim 18, wherein a tip of the bronchoscope has a tip positioning device, and the tip positioning device is for generating a positioning signal and transmitting the positioning signal to the processing device.

20. The bronchoscope path display device according to claim 19, wherein the tip positioning device is selected from the group consisting of accelerometer and a gyroscope.
